SEQUENCE LISTING (42 sequences)

SEQ ID NO: 1 - DNA, 101 bp, Lactuca sativa
  source 1..101: mol_type = genomic DNA
gacagcctttgtgacaacccsyttttcttggtgaaaacaaacccttttttcgtgaaagacaatttgttgtgttttttactttatgttctttatttrcttag

SEQ ID NO: 2 - DNA, 101 bp, Lactuca sativa
  source 1..101: mol_type = genomic DNA
gacagcctttgtgacaacccsyttttcttggtgaaaacaaacccttttttggtgaaagacaatttgttgtgttttttactttatgttctttatttrcttag

SEQ ID NO: 3 - DNA, 101 bp, Lactuca sativa
  source 1..101: mol_type = genomic DNA
amtgagctttctaactaatagcataatcgttgaaagtaaacacataccytgtaataaatcttcatttatccaaatccttagcatgatctkaattaacatat

SEQ ID NO: 4 - DNA, 101 bp, Lactuca sativa
  source 1..101: mol_type = genomic DNA
amtgagctttctaactaatagcataatcgttgaaagtaaacacataccytataataaatcttcatttatccaaatccttagcatgatctkaattaacatat

SEQ ID NO: 5 - DNA, 101 bp, Lactuca sativa
  source 1..101: mol_type = genomic DNA
atattttcccgagaactaaatttagaccattagatcaaaaaaaatggagggcagtgattttttatggcagttttataaataactagaaaaaagagatgggg

SEQ ID NO: 6 - DNA, 101 bp, Lactuca sativa
  source 1..101: mol_type = genomic DNA
atattttcccgagaactaaatttagaccattagatcaaaaaaaatggaggacagtgattttttatggcagttttataaataactagaaaaaagagatgggg

SEQ ID NO: 7 - DNA, 101 bp, Lactuca sativa
  source 1..101: mol_type = genomic DNA
ggactcctgctcaagcatcactcgcatgggtggcggaacaacggtggtataggtatggctcgaattcttgtggtggaggaaaggaatgcggtcactataca

SEQ ID NO: 8 - DNA, 101 bp, Lactuca sativa
  source 1..101: mol_type = genomic DNA
ggactcctgctcaagcatcactcgcatgggtggcggaacaacggtggtatcggtatggctcgaattcttgtggtggaggaaaggaatgcggtcactataca

SEQ ID NO: 9 - DNA, 101 bp, Lactuca sativa
  source 1..101: mol_type = genomic DNA
caccccaaaaamtccaaataatcayccttcaaatcacaaaagaaactttcataccaatcattctttgaatttcawtctaaaccrattattaacccttcgaa

SEQ ID NO: 10 - DNA, 101 bp, Lactuca sativa
  source 1..101: mol_type = genomic DNA
caccccaaaaamtccaaataatcayccttcaaatcacaaaagaaactttcgtaccaatcattctttgaatttcawtctaaaccrattattaacccttcgaa

SEQ ID NO: 11 - DNA, 101 bp, Lactuca sativa
  source 1..101: mol_type = genomic DNA
cgcaaaccrcaccgtaaagtgcaggatatcaaaaacatgaaccgcgaaccgyaccaagaatgttytaaaccgcattacacggtgcaggcggtttatgtkgt

SEQ ID NO: 12 - DNA, 101 bp, Lactuca sativa
  source 1..101: mol_type = genomic DNA
cgcaaaccrcaccgtaaagtgcaggatatcaaaaacatgaaccgcgaaccayaccaagaatgttytaaaccgcattacacggtgcaggcggtttatgtkgt

SEQ ID NO: 13 - DNA, 101 bp, Lactuca sativa
  source 1..101: mol_type = genomic DNA
gtagcttgattcatgtgatgagatcttatctataccctatacaggttgttatgaatgacaattgacttttatcacatccttatgaaagtyccatagtatgt

SEQ ID NO: 14 - DNA, 101 bp, Lactuca sativa
  source 1..101: mol_type = genomic DNA
gtagcttgattcatgtgatgagatcttatctataccctatacaggttgttctgaatgacaattgacttttatcacatccttatgaaagtyccatagtatgt

SEQ ID NO: 15 - DNA, 101 bp, Lactuca sativa
  source 1..101: mol_type = genomic DNA
atcttggtagaattcacaagattcatcatgacatcaacttgtaccctaagagaaagtatatagtctaatgcttctttaaagatggtgaaatcatccatggc

SEQ ID NO: 16 - DNA, 101 bp, Lactuca sativa
  source 1..101: mol_type = genomic DNA
atcttggtagaattcacaagattcatcatgacatcaacttgtaccctaagggaaagtatatagtctaatgcttctttaaagatggtgaaatcatccatggc

SEQ ID NO: 17 - DNA, 101 bp, Lactuca sativa
  source 1..101: mol_type = genomic DNA
acatgttcaaaattcggtayggaattaacacgatttaacattgttgtcgtatttttcgtatttgtcgtgttatgtatgattargtattaattaattaaact

SEQ ID NO: 18 - DNA, 101 bp, Lactuca sativa
  source 1..101: mol_type = genomic DNA
acatgttcaaaattcggtayggaattaacacgatttaacattgttgtcgtgtttttcgtatttgtcgtgttatgtatgattargtattaattaattaaact

SEQ ID NO: 19 - DNA, 101 bp, Lactuca sativa
  source 1..101: mol_type = genomic DNA
ggtgtaataattctttttcacatgtagtttcatcttcttgtggcactttgagtgatatttttkctatttgggatctagatcgtatttctcataaaaggatg

SEQ ID NO: 20 - DNA, 101 bp, Lactuca sativa
  source 1..101: mol_type = genomic DNA
ggtgtaataattctttttcacatgtagtttcatcttcttgtggcactttgggtgatatttttkctatttgggatctagatcgtatttctcataaaaggatg

SEQ ID NO: 21 - DNA, 101 bp, Lactuca sativa
  source 1..101: mol_type = genomic DNA
caagaataacaagtratctctaacatggtggaactaatcttcctggtggcctgttctcgaagaatcttagccctctctaactcaacctgagcttgttgcct

SEQ ID NO: 22 - DNA, 101 bp, Lactuca sativa
  source 1..101: mol_type = genomic DNA
caagaataacaagtratctctaacatggtggaactaatcttcctggtggcttgttctcgaagaatcttagccctctctaactcaacctgagcttgttgcct

SEQ ID NO: 23 - DNA, 101 bp, Lactuca sativa
  source 1..101: mol_type = genomic DNA
ctcatatatgyggtcattgttgagtaaggattggttagaattgtagattagttgtcttytcttttctctttggtgtcttcsatcatgatttcttttgctcy

SEQ ID NO: 24 - DNA, 101 bp, Lactuca sativa
  source 1..101: mol_type = genomic DNA
ctcatatatgyggtcattgttgagtaaggattggttagaattgtagattaattgtcttytcttttctctttggtgtcttcsatcatgatttcttttgctcy

SEQ ID NO: 25 - DNA, 101 bp, Lactuca sativa
  source 1..101: mol_type = genomic DNA
ttgccggttcgcatccaagaggtgatgcccaacragcaacttcatcygaagttgctcccactccatagactctagatttataaaaawtgacttttagaccc

SEQ ID NO: 26 - DNA, 101 bp, Lactuca sativa
  source 1..101: mol_type = genomic DNA
ttgccggttcgcatccaagaggtgatgcccaacragcaacttcatcygaatttgctcccactccatagactctagatttataaaaawtgacttttagaccc

SEQ ID NO: 27 - DNA, 101 bp, Lactuca sativa
  source 1..101: mol_type = genomic DNA
tatattcatgattagycgaygrytgktttgggaaaccaggttrgaggacyatcagtaggactcgagtgagtcaagatgywgattgagtgaatgtccrrcat

SEQ ID NO: 28 - DNA, 101 bp, Lactuca sativa
  source 1..101: mol_type = genomic DNA
tatattcatgattagycgaygrytgktttgggaaaccaggttrgaggacygtcagtaggactcgagtgagtcaagatgywgattgagtgaatgtccrrcat

SEQ ID NO: 29 - DNA, 101 bp, Lactuca sativa
  source 1..101: mol_type = genomic DNA
gttycctcactrggwttagtrggtytaaggcactaatgtyggaccttattagttatgataggatgttaggagtttgcatgacatttgcatgtgttatgtgt

SEQ ID NO: 30 - DNA, 101 bp, Lactuca sativa
  source 1..101: mol_type = genomic DNA
gttycctcactrggwttagtrggtytaaggcactaatgtyggaccttattggttatgataggatgttaggagtttgcatgacatttgcatgtgttatgtgt

SEQ ID NO: 31 - DNA, 101 bp, Lactuca sativa
  source 1..101: mol_type = genomic DNA
agtgagccaatatcatgcacaaaagcatggaaacaaaacatataygtacactatgtcaagtcatgtaatcagatcaatccatacarcatacatcgataact

SEQ ID NO: 32 - DNA, 101 bp, Lactuca sativa
  source 1..101: mol_type = genomic DNA
agtgagccaatatcatgcacaaaagcatggaaacaaaacatataygtacattatgtcaagtcatgtaatcagatcaatccatacarcatacatcgataact

SEQ ID NO: 33 - DNA, 101 bp, Lactuca sativa
  source 1..101: mol_type = genomic DNA
catcgtgtctttcaataacttccaccgacttaaaagtttggtctttagttatctgactgcaaatactgtcttttgcttatcgctayagttgcagctttcaa

SEQ ID NO: 34 - DNA, 101 bp, Lactuca sativa
  source 1..101: mol_type = genomic DNA
catcgtgtctttcaataacttccaccgacttaaaagtttggtctttagttgtctgactgcaaatactgtcttttgcttatcgctayagttgcagctttcaa

SEQ ID NO: 35 - DNA, 101 bp, Lactuca sativa
  source 1..101: mol_type = genomic DNA
gtcatttcatcatcaattgcttctttccaaaatattacatcttgagaccgtrtggcctcttgaaatgtctttggatcatccccaatactrkaamaatwaga

SEQ ID NO: 36 - DNA, 101 bp, Lactuca sativa
  source 1..101: mol_type = genomic DNA
gtcatttcatcatcaattgcttctttccaaaatattacatcttgagaccgcrtggcctcttgaaatgtctttggatcatccccaatactrkaamaatwaga

SEQ ID NO: 37 - DNA, 101 bp, Lactuca sativa
  source 1..101: mol_type = genomic DNA
ggtcagatctgatcggrctaagatcttaagatcctacttgggtcctaagactctagtatagaacctagtaagattgatatcgtttttyacttctaaaattt

SEQ ID NO: 38 - DNA, 101 bp, Lactuca sativa
  source 1..101: mol_type = genomic DNA
ggtcagatctgatcggrctaagatcttaagatcctacttgggtcctaagattctagtatagaacctagtaagattgatatcgtttttyacttctaaaattt

SEQ ID NO: 39 - DNA, 101 bp, Lactuca sativa
  source 1..101: mol_type = genomic DNA
atttcataccttttccatgggctaccccatggtcttcccttgtagtgccttggctaccctgargtcatatacactagttccatgggatacccccatggtca

SEQ ID NO: 40 - DNA, 101 bp, Lactuca sativa
  source 1..101: mol_type = genomic DNA
atttcataccttttccatgggctaccccatggtcttcccttgtagtgcctcggctaccctgargtcatatacactagttccatgggatacccccatggtca

SEQ ID NO: 41 - DNA, 101 bp, Lactuca sativa
  source 1..101: mol_type = genomic DNA
tgatggttcytcttcaagtggaaccaaaggtgttcttcctactccctctatcaaacctaaagaagctraatgcttctattgtcatgacaaactatattgtc

SEQ ID NO: 42 - DNA, 101 bp, Lactuca sativa
  source 1..101: mol_type = genomic DNA
tgatggttcytcttcaagtggaaccaaaggtgttcttcctactccctctaccaaacctaaagaagctraatgcttctattgtcatgacaaactatattgtc

(19) United States
(12) Patent Application Publication
(10) Pub. No.: US 2026/0198439 A1
(43) Pub. Date: Jul. 16, 2026

(54) No Bleed Lettuce

(71) Applicant: Bejo Zaden B.V., Warmenhuizen (NL)

(72) Inventors: Rens Bogers, Warmenhuizen (NL); Stefanus Johannes Kaandorp, Warmenhuizen (NL); Teunis Scheurwater, Warmenhuizen (NL); Roelof Marinus Veenstra, Warmenhuizen (NL); Albertus Johannes Maria Schrijver, Warmenhuizen (NL)

(21) Appl. No.: 19/133,313

(22) PCT Filed: Nov. 29, 2022
(86) PCT No.: PCT/EP2022/083621

§ 371 Date: May 28, 2025

(51) Int. Cl. A01H 6/14; A01H 5/12

(52) U.S. Cl. CPC A01H 6/1472; A01H 5/12

(57) ABSTRACT

Provided herein are lettuce plants having a no bleed phenotype, i.e. the lettuce head thereof does not substantially secrete exudate from the cut surface after cutting the lettuce head from the root system the phenotype being obtainable, obtained, or is from a lettuce plant deposited as NCIMB 43972.

### CROSS-REFERENCE TO RELATED APPLICATION

This application is the United States national phase of International Patent Application No. PCT/EP2022/083621 filed Nov. 29, 2022, the disclosure of which is hereby incorporated by reference in its entirety.

### REFERENCE TO A SEQUENCE LISTING

The Sequence Listing associated with this application is filed in electronic format via Patent Center and is hereby incorporated by reference into the specification in its entirety. The name of the file containing the Sequence Listing is 2501986.xml. The size of the file is 41,265 bytes, and the file was created on May 21, 2025.

### BACKGROUND OF THE INVENTION

### Field of the Invention

The present invention relates to lettuce plants having a no bleed phenotype, i.e. the lettuce head thereof does not substantially secrete exudate from the cut surface after cutting the lettuce head from the root system. The present invention further relates to seeds, progeny, edible parts, egg cells, callus, suspension culture, somatic embryos, clones, embryos, or plant parts of the present lettuce plants and to methods for identifying the present lettuce plants.

Lettuce, also referred to as Lactuca sativa, is a cultivated plant that belongs to the daisy family (Asteraceae). This family comprises over 23,000 species with a broad geographical distribution from the tropics to the polar region. The Asteraceae family includes, besides lettuce, several other food crops such as chicory, endive, sunflower and artichoke.

Apart from lettuce, the genus Lactuca contains roughly 75 wild species. Although these species have undesirable agronomic traits, such as a bitter flavour, a high latex content, or leaf spines, they can be used to improve, for example, the taste, texture, and disease resistance of cultivated lettuce. The three wild species Lactuca saligna L., Lactuca serriola L. and Lactuca virosa L are frequently used in cultivated lettuce breeding programs.

Lettuce is an annual crop mostly grown as a leaf vegetable. Lettuce produces crispy leaves and has an average size of 15-30 cm. The leaves are usually green, but red and other colours are known too. Leaves can also have various shapes and textures. Breeding efforts have resulted in many different varieties of lettuce subdivided into seven different morphological types.

The crisphead group contains iceberg and batavia lettuce types. Iceberg lettuce has a large, firm head with a crisp texture and a white or creamy yellow interior. Batavia lettuce resembles the iceberg type but has a smaller and less firm head. The butterhead group has a small, soft head with an almost oily texture. The romaine, also known as cos lettuce, has elongated upright leaves forming a loose, loaf-shaped head and the outer leaves are usually dark green. Leaf lettuce, also known as cutting lettuce, comes in many varieties that do not form a head. Latin lettuce has a loose head with thick leathery leaves, dark green colour, and looks like a cross between romaine and butterhead. Stem lettuce has long, narrow leaves and thick, edible stems. Oilseed lettuce is a type grown for its large seeds that contain a high percentage of oil. Because of the bitter taste of its leaves, this type is not eaten as a vegetable.

Lettuce is grown commercially wherever environmental conditions permit the production of an economically viable yield. Important markets are the USA, China and the EU. Lettuce is an increasingly popular crop as worldwide lettuce consumption continues to increase.

In order to be sold to the consumer, the lettuce plant needs to be harvested. During harvest a cut is made to separate the lettuce head from the root system. This cut disrupts the continuity of the plant tissue and a fluid (exudate) comes out of the cut surface.

Phenotypically, it appears as if the plant is bleeding, hence the breeders often refer to this phenomenon as “bleeding of lettuce”. The exudate of freshly cut lettuce plants is initially white and milky and turns brown at a later stage. The components of this fluid are: (1) xylem sap or xylem fluid, (2) phloem sap or phloem fluid and (3) contents of the disrupted cells. Xylem sap consists primarily of a watery solution of hormones, mineral elements and other nutrients. Phloem sap consists primarily of sugars, hormones, and mineral elements dissolved in water. It flows from where carbohydrates are produced or stored (sugar source) to where they are used. The contents of the cells that are mechanically disrupted supply enzymes to this mixture.

When the exudate is exposed to air it undergoes a series of chemical reactions over time that result in discoloration, i.e. the initially white, milky fluid that bleeds out of the surface of the cut turns brown. A substantial part of the discoloration or browning is a result of enzymatic oxidation. The words discoloration and browning will be used interchangeably. The oxidation is caused among others by polyphenol oxidase. Products of this reaction are brown pigments. Vegetables are rich in polyphenols that serve as substrates for the enzymatic oxidation reactions. Oxygen from air is required for this reaction.

Products of the oxidation reaction can further undergo polymerization. The resulting insoluble polymers are often brown. In the case of lettuce plants, the substrates of the oxidation reaction are tyrosine, caffeic acid and chlorogenic acid.

Bleeding and subsequent discoloration resulting from it have a negative impact on the quality of the end product. A lettuce plant, e.g. iceberg, with a brown stain along the area of the cut is visually less appealing to the consumer because it does not look fresh. The harvested lettuce heads are often packed in plastic foil directly after harvesting. Since the heads are still fresh the juices distribute in the plastic package, resulting in brown, sticky juices at the inside of the plastic package, which is making the product less appealing to the consumer. Moreover, the juices that bleed out of the cut are rich in nutrients, such as sugars, and this creates an optimal environment for microorganisms to grow, which can compromise food safety.

### DESCRIPTION OF RELATED ART

There are several known approaches to counter the discoloration of lettuce after cutting. Perhaps the oldest solution is one that is still used in some Mediterranean countries: the freshly cut lettuce head is sprinkled with freshly squeezed lemon juice. A modern counterpart for this approach is adding vitamin C as a reducing agent. Adding vitamin C or other chemicals (EDTA, cyclodextrin, L-cysteine) may alter the taste of lettuce and does not solve the food safety issues.

A second approach is blanching. The plant is shortly exposed to high temperatures with the aim of inactivating the oxidating enzymes that contribute to discoloration. This might result in change of taste, texture and color, which can influence the perception of freshness of the product by the consumer and it is an additional procedure that needs to be performed.

A third approach to prevent coloration is that the freshly harvested lettuce can be packed at a low temperature in a low oxygen environment. This is mostly suitable for pre-cut lettuce. Indeed, the enzymatic browning can be significantly reduced in this way. However, the modified, low oxygen environment promotes anaerobic fermentation and in turn the end product has an off-odor and the lettuce does not appear fresh to the customer.

There is a series of patent literature documents disclosing lettuce plants that show reduction of (wound-induced) discoloration and methods for screening for the presence of this trait in lettuce: EP2428112, EP1973396, EP1988764, EP3419416.

Further, Rijk Zwaan is marketing lettuce with the Knox™ trait-lettuce that exhibits slow discoloration of cut edges. Knox™ Core, also from Rijk Zwaan, is a different trait: slower discoloration of the cut edge of a head of lettuce. The head appears fresh for a longer time.

These approaches address the problem of discoloration but do not solve the problem of bleeding of the lettuce head after cutting, i.e., such plants still bleed but the fluid does not turn brown or the process is decelerated. The disadvantage of this approach is that the lettuce plant, when placed in a plastic bag, still secretes fluids from the cut and the fluids distribute on the surface of the plastic packaging. After a few days the fluid becomes sticky and lettuce removed from such plastic packaging will not appear fresh or otherwise visually appealing to the end customer. Moreover, the nutrient rich juices promote growth of microorganisms and compromise food safety.

There is thus a need in this technical field to provide lettuce plants that do not bleed, i.e., lettuce plants that substantially do not exert juices (exudate) from the surface of the cut, after a cut is made to separate the head of lettuce from the root system of the plant.

### SUMMARY OF THE INVENTION

It is an object of the present invention, amongst other objects, to meet the above need in the art.

According to the present invention, the above need is met as outlined in the appended claims.

Specifically, the above need is met by the present invention by providing lettuce plants having a phenotype that the lettuce head thereof does not substantially secrete exudate from the cut surface after cutting the lettuce head from the root system, the phenotype is obtainable, obtained, or is from a lettuce plant deposited as NCIMB 43972.

The present inventors have surprisingly identified lettuce plants that do not bleed from the surface of the cut when a cut is made to separate the lettuce head from root system. The plants according to the present invention were compared, and show to differ, with commercially available lettuce plants, i.e., plants that exert discoloration and plants with Knox™ core and Knox™ traits. All plants were tested under similar growth conditions. It is noted that the differences observed in bleeding from the surface of the cut between the plants of the present invention and prior art plants do not result in a difference in turgor pressure or otherwise different water need of the plants.

Within the context of the present invention, lettuce plants comprising the ‘No bleed’ trait, or phenotype, are plants that substantially do not exert juices (exudate) from the surface of the cut, after a cut is made to separate the head of lettuce from the root system of the plant. Plants that score 1 or 2, on a scale of 1 to 9, according to the protocol for evaluation of the bleeding-see Example 1 for evaluation of the bleeding) are considered plants that substantially do not exert juices (exudate) from the cut, after a cut is made to separate the head of lettuce from the root system of the plant.

Representative seeds of plants according to the invention, Lettuce plants, Lactuca sativa 16795-02C0-620, deposit NCIMB 43972, were deposited at NCIMB Limited, Craibstone Estate, 35 Ferguson Building, Bucksburn, Aberdeen AB21 9YA, United Kingdom on Apr. 13, 2022.

According to the present invention, the term “substantially” comprises that traces of exudate can be observed at the cut surface. Lettuce plants showing traces of exudate at the cut surface are considered, within the context of the present invention, to have a no bleed phenotype.

The present no bleed phenotype is encoded by two or more genes.

According to the present invention, a first gene encoding the no bleed phenotype is located in a first genomic region on chromosome 5 at a position corresponding with position 189,391,684 bp to 201,101,671 bp of the lettuce reference genome V8 (V8 (Reyes-Chin-Wo et al., 2017; https://lgr.fenomecenter.ucdavis.edu/)

Preferably, the first genomic region comprises one or more sequences selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17 and SEQ ID No. 19.

According to the present invention, the present phenotype is preferably genetically linked to one or more sequences selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17 and SEQ ID No. 19.

According to the present invention, a second gene encoding the no bleed phenotype is located in a second genomic region on chromosome 8 at a position corresponding with position 44,786,623 bp to 62,697,887 bp of the lettuce reference genome V8.

The second genomic region preferably comprises one or more sequences selected from the group consisting of SEQ ID No. 21, SEQ ID No. 23, SEQ ID No. 25, SEQ ID No. 27, SEQ ID No. 29, SEQ ID No. 31, SEQ ID No. 33, SEQ ID No. 35, SEQ ID No. 37, SEQ ID No. 39 and SEQ ID No. 41.

According to the present invention, the no bleed phenotype is preferably genetically linked to one or more sequences selected from the group consisting of SEQ ID No. 21, SEQ ID No. 23, SEQ ID No. 25, SEQ ID No. 27, SEQ ID No. 29, SEQ ID No. 31, SEQ ID No. 33, SEQ ID No. 35, SEQ ID No. 37, SEQ ID No. 39 and SEQ ID No. 41.

According to an especially preferred embodiment of the present invention, the above first gene is homozygously present in the genome of the lettuce plant and/or the above second gene is homologously present in the genome of the lettuce plant.

Preferably, the present lettuce plants having the no bleed phenotype also display resistances to Xanthomonas campestris, Erwinia carotovora, Pseudomonas cichorii, Pseudomonas viridiflava, Alternaria sonchi, Bremia lactucae, Sclerotinia, Fusarium oxysporum, Pythium, Rhizorhapis suberifaciens, Lettuce Mosaic Virus (LMV), Tomato Spotted Wilt Virus (TSWV), Lettuce Big Vein Virus (LBVV), Lettuce dieback associated viruses and/or Nasonovia ribisnigri. These resistances can be suitably combined with the no bleed phenotype without decreasing the vigour or economic value of the present plants.

Considering the above, the present invention also relates to seeds, progeny, edible parts, egg cells, callus, suspension culture, somatic embryos, clones, embryos, or plant parts of, or derived from, the lettuce plants described above.

Additionally, the present invention relates to method for identifying lettuce plants comprising a phenotype that lettuce head thereof does not substantially secrete exudate from the cut surface after cutting the lettuce head from the root system, the method comprises the steps of:
identifying the presence of a first gene by establishing the presence in the genome of the lettuce plant of one or more markers selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17 and SEQ ID No. 19; and/OR
identifying the presence of a second gene by establishing the presence in the genome of the lettuce plant of one or more markers selected from the group consisting of SEQ ID No. 21, SEQ ID No. 23, SEQ ID No. 25, SEQ ID No. 27, SEQ ID No. 29, SEQ ID No. 31, SEQ ID No. 33, SEQ ID No. 35, SEQ ID No. 37, SEQ ID No. 39 and SEQ ID No. 41.
   Establishing, or identifying, the presence of markers in the genome of lettuce plants is common practice in the art for years. For example, genomic DNA can be isolated and subsequently subjected to nucleic acid amplification using appropriate primers. Amplified DNA fragments can be visualized using gels are detected using a dedicated apparatus.

Preferably, the present method further comprises the step of establishing the homozygous presence of the first gene and/or the second gene. This can, for example, be accomplished by detecting the presence of SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20; and/or SEQ ID No. 22, SEQ ID No. 24, SEQ ID No. 26, SEQ ID No. 28, SEQ ID No. 30, SEQ ID No. 32, SEQ ID No. 34, SEQ ID No. 36, SEQ ID No. 38, SEQ ID No. 40, SEQ ID No. 42. Detecting these markers indicates that either the first gene or the second gene is not homozygously present.

According to yet another preferred embodiment, the present invention relates to use of one or more of genomic sequences, or nucleic acid sequences, comprising one or more nucleic acid sequences selected from the group consisting selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17 and SEQ ID No. 19, SEQ ID No. 21, SEQ ID No. 23, SEQ ID No. 25, SEQ ID No. 27, SEQ ID No. 29, SEQ ID No. 31, SEQ ID No. 33, SEQ ID No. 35, SEQ ID No. 37, SEQ ID No. 39 and SEQ ID No. 41, for identifying or providing a lettuce plant having a phenotype that the lettuce head thereof does not substantially secrete exudate from the cut surface after cutting the lettuce head from the root system.

According to a yet another aspect the present invention relates to the nucleic acid sequence selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17 and SEQ ID No. 19, SEQ ID No. 21, SEQ ID No. 23, SEQ ID No. 25, SEQ ID No. 27, SEQ ID No. 29, SEQ ID No. 31, SEQ ID No. 33, SEQ ID No. 35, SEQ ID No. 37, SEQ ID No. 39 and SEQ ID No. 41.

The present invention will be further detailed in the examples below. In the examples, reference is made to figures wherein:

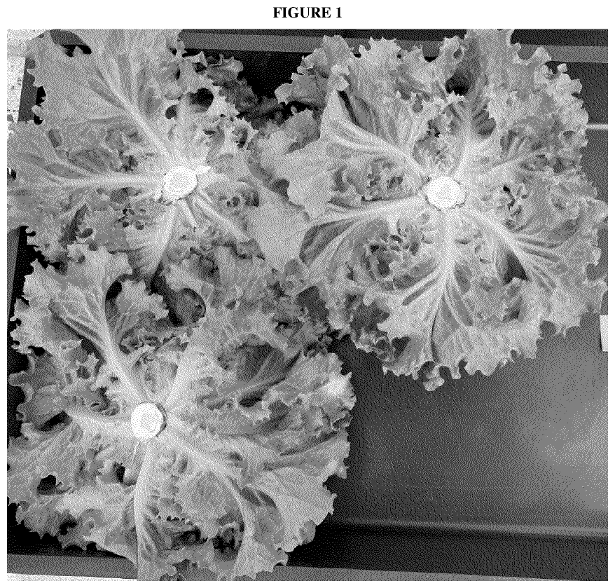

FIGS. 1 to 3: show lettuce variety Meditation being a Knox™ variety. The Knox™ varieties exhibit a delay of piking (discoloration) of cut leaf surfaces;

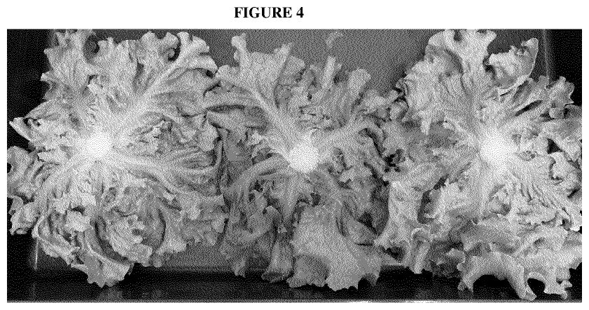

FIGS. 4 to 6: show plants according to the invention (NCIMB 43972) comprising the “No bleed” trait, batavia type;

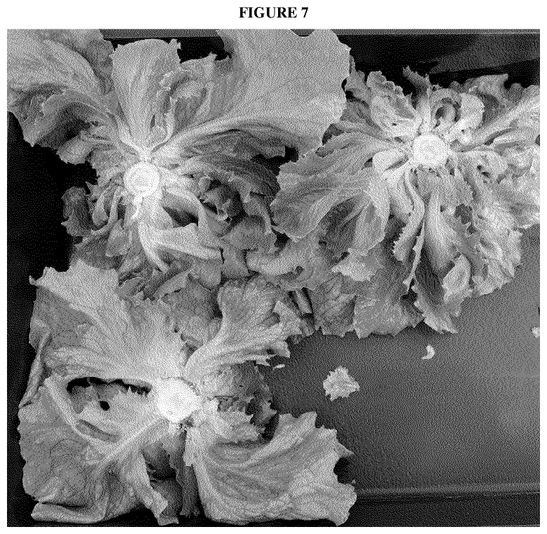

FIGS. 7 to 9: show Bandanas being a Knox™ core variety. Knox™ core varieties show slowed discoloration of the cut edge of a head of lettuce. The head maintains fresh appearance for a longer time as compared to conventional lettuce plants.

### BRIEF DESCRIPTION OF THE DRAWINGS

### FIG. 1.

Three lettuce plants were assessed within minutes after cutting. It can be observed that even though the heads are turned upside down there is a significant amount of white fluid (exudate) that accumulates on the surface of the cut. The fluid is white and milky and accumulates on the peripheries of the plane of the cut. In the beginning the exudate is white and does not have the undesired brown color. These plants score 9 for bleeding (see Example 1 for the scale of bleeding).

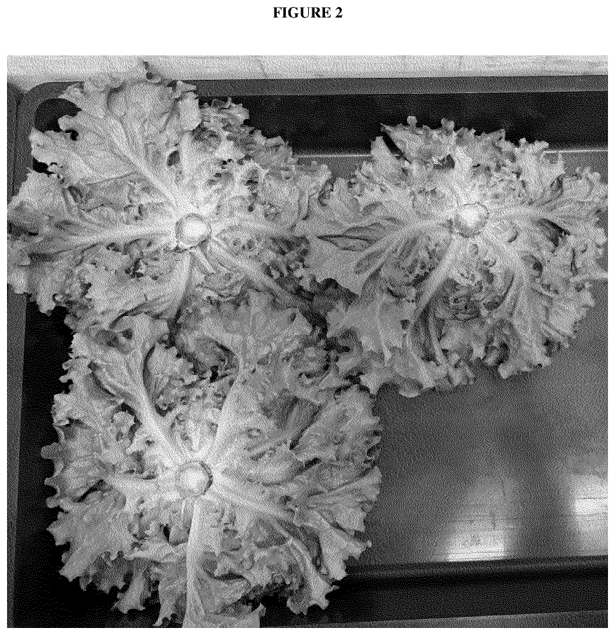

### FIG. 2.

The lettuce plants from FIG. 1 were assessed 1 day after cutting. The areas of the cut that were covered with exudate the day before, were dry at the point of evaluation and they have discolored into brown “stains”. This corresponded to high browning ((++); see Example 1 for the scale of browning).

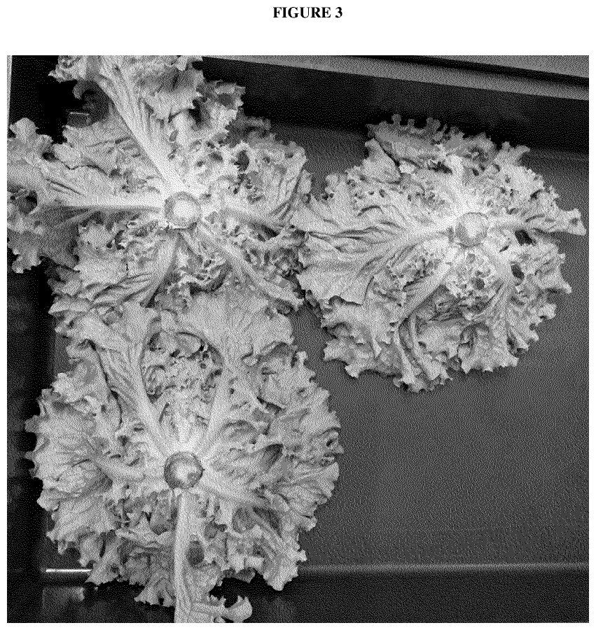

### FIG. 3.

The lettuce plants from FIGS. 1 and 2 were assessed six days after cutting. The areas of the cut were dry and the areas that were previously covered with exudate (ref. FIG. 1) were now very significantly discolored and turned brown ((+++); see Example 1 for the scale of browning).

### FIG. 4.

Three lettuce plants according to the invention. Picture taken within a few minutes after cutting. The cut areas were not covered with exudate, in contrast to FIG. 1. Some areas displayed smaller amounts of transparent fluid or slight amounts of exudate. Plants that remained predominantly dry, scored 1 for bleeding. Plants that showed some accumulation of exudate scored 2 for bleeding.

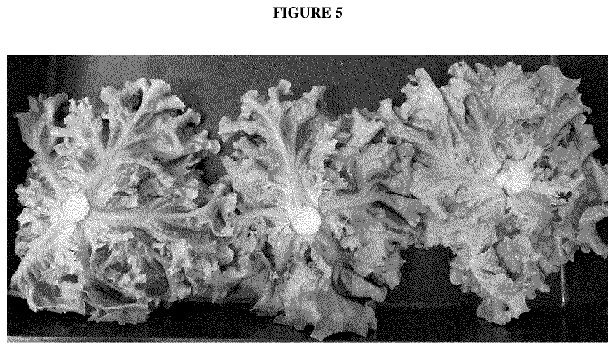

### FIG. 5.

The same plants as in FIG. 4 were assessed for browning, one day after cutting. The surface of the cut was dry and there was no discoloration observed. A possible explanation is that because there was no exudate accumulated, it could not have undergone discoloration and turned brown. These plants score (−) for browning, see Example 1 for details.

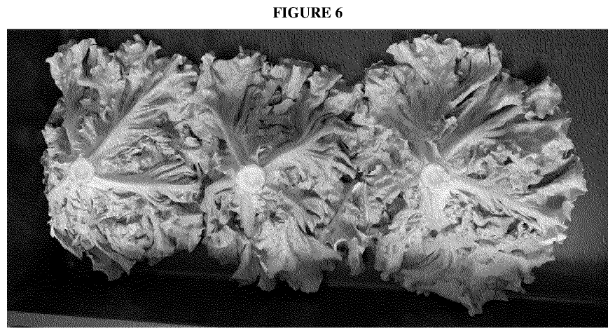

### FIG. 6.

The same plants as in FIGS. 4 and 5 were assessed for browning/discoloration, six days after cutting. The surface of the cut was predominantly dry and there was only a slight discoloration or browning of the cut head observed. Interestingly, the discoloration occurred mostly in the center of the cut area and not in the peripheral areas as in the case of FIGS. 2 and 3 (in plants that bleed). The plants in this figure scored (−/+) for browning/discoloration; see Example 1 for the scale of browning.

### FIG. 7.

Three plants of the variety Bandanas were assessed within a few minutes after cutting. A significant amount of exudate accumulated on the surface of the cut. The fluid was white and was present at the periphery of the cut. These plants score 9 for bleeding.

In the beginning, within a few minutes after cutting, the exudate was white and did not have the undesired brown color.

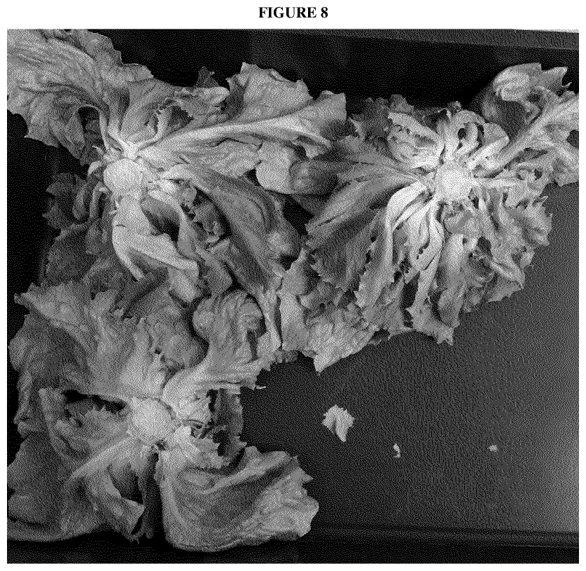

### FIG. 8.

The same plants as in FIG. 7 were assessed for discoloration/browning, one day after cutting. The areas of the cut that were covered with exudate the day before, were dry at the point of evaluation and they had to a certain degree discolored into brown “stains”. This corresponded to slight browning (a score (+/−), see Example 1 for the scale of browning). Interestingly, as in the case of plants from FIG. 1-3, not comprising the no bleed trait, the browning was observed mostly at the periphery of the cut, in the places that had exerted white fluid just after the cut. The browning or discoloration was much less pronounced or absent in the center of the plant.

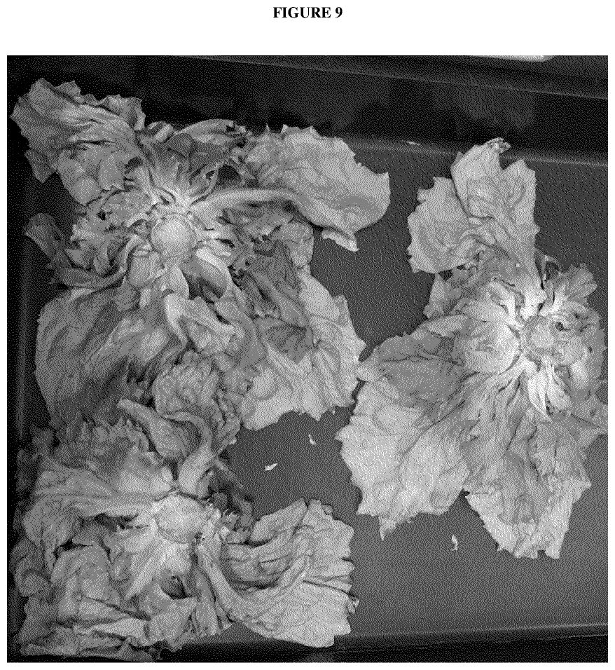

### FIG. 9.

The same plants as in FIG. 7 and FIG. 8 were assessed for discoloration/browning, six days after cutting. The areas of the cut were mostly dry and the areas that had previously white fluid (exudate ref. FIG. 1) were very significantly discolored at the point of evaluation and turned brown (+++), see Example 1 for the scale of browning. In two out of three plants it was easy to observe that the discoloration or browning was much more pronounced at the periphery of the cut and not in the center of the plant. This is similar to what was observed for plants not comprising the “No bleed” trait (ref. FIG. 3) and is a different pattern of discoloration/browning as plants comprising the trait, where the discoloration at day six mostly occurs in the center of the cut (ref. FIG. 6)

### DESCRIPTION OF THE INVENTION

### Examples

### Example 1: Assessment of the “No Bleed” Trait and Browning

Different lettuce varieties and the plant according to the invention were grown from seed—see Table 1. Germination took place in a climate room and seedlings were subsequently moved to the greenhouse in black plastic pots of 20×10×10 cm (height×width×depth) filled with peat. Plants were watered daily using drip irrigation containing nutrients. In the greenhouse the temperature settings were 20 degrees Celsius in combination with an average RH (relative humidity) of 60% during the day (16 hours) and 12 degrees Celsius with an RH of 90% (8 hours) during night. All plants were grown in the same conditions.

Ten weeks after sowing, the plants were harvested and evaluated. The plants were harvested individually by cutting the lettuce head manually using a knife. In practice, this resulted in a flat cutting surface. After cutting each lettuce the head was turned upside down and each plant was evaluated individually by three observers and a phenotypic score was agreed on.

The first evaluation was performed within a few minutes from harvesting and at this moment only the “bleeding” was evaluated (see Table 1: score bleed fresh). After harvesting the plants were stored in a cold room (4 degrees Celsius). Here the second and third evaluation took place after respectively one and six days after harvesting. Only the extent of browning was evaluated (see Table 1: “Score browning 1 day after harvest” and “Score browning 6 days after harvest”). For each measurement at least three plants were assessed.

Evaluation took place by visual inspection of the harvested lettuce heads. For the phenomenon of bleeding the amount of juices that were exerted from the cut surface (exudate) was observed and a score from 1 to 9 was given. A score of 1 means no bleeding: the surface of the cut remains dry. A score of 2 means predominantly no bleeding with traces of exudate present at some places. A score of 9 means full bleeding: the cut surface exerted juices (exudates) abundantly. Bleeding was evaluated just after cutting (harvest).

For the evaluation of browning a scale from (−), though (−/+), (+), (++) up until (+++) was used. A score of (−) means that no browning or any form of discoloration as compared to a fresh cut was observed; (−/+) means little browning was observed. A score of (++) means significant browning. A score of (+++) corresponds to the maximal browning of surface of the cut observed. Browning was evaluated 1 day and 6 days after cutting (harvest).

TABLE 1
Evaluation of bleeding and browning/discoloration.

| | | Score | Score browning 1 | Score browning 6 |
|---|---|---|---|---|
| Name | Type | bleed fresh | day after harvest | days after harvest |
| NCIMB 43972 | Batavia | 1.8 | (−) | (−/+) |
| Meditation* | Batavia | 9 | (++) | (+++) |
| Bandanas** | Iceberg | 9 | (−/+) | (+++) |
| Malis | Butterhead | 9 | (+++) | NA |
| Starena | Mini Romaine | 9 | (++) | NA |

*Meditation is a Knox ™ variety. The Knox ™ varieties exhibit a delay of piking (discoloration) of cut leaf surfaces.
**Bandanas is a Knox ™ core variety. Knox ™ core varieties show slowed discoloration of the cut edge of a head of lettuce. The head maintains fresh appearance for a longer time as compared to conventional lettuce plants.

### Example 2: Pictures of the Assessment of the “No Bleed” Trait

Two commercial varieties and the plant according to the invention (the deposit NCIMB 43972) were compared and assessed for the “no bleed” trait and subsequent browning of the cut surface. Meditation is a batavia type lettuce and does not comprise the “No bleed” trait, FIGS. 1-3. Mediatation is a Knox™ variety. The Knox™ varieties exhibit a delay of piking (discoloration) of cut leaf surfaces.

### Example 3: Genetic Mapping of the No Bleed Trait

To study the inheritance and genetics behind the No bleed trait the phenotype of plants from two mapping populations was determined and the plants were genotyped using KASP markers.

Population 1: P1 (a Lettuce Line Containing the No Bleed Trait)×P2 (a Lettuce Line without the No Bleed Trait).

An F1S1 population of 213 plants derived from P1 and P2 segregated for the No Bleed trait. 18 plants had a phenotype comparable to P1. 139 Plants had a phenotype comparable to P2. The other 56 plants had an intermediate phenotype. The population was genotyped using 298 KASP markers covering the whole genome. QTL analysis was performed with mapQTL 6 using standard interval mapping. A QTL of 10.6 cM with a LOD score of 45 was identified around 200 Mbp on chromosome 5. The borders of this QTL are 189.391.684 and 201.101.671 bp. This QTL explained 62 percent of the phenotypes.

Population 2: P1 (a Lettuce Line Containing the No Bleed Trait)×P3 (a Lettuce Line without the No Bleed Trait).

An F1S1 population derived from P1 and P3 segregated for the No bleed trait. From the 398 plants, 186 showed a phenotype comparable to P1, 88 plants showed an intermediate phenotype and 124 showed a phenotype comparable to P3. 134 KASP markers were used to genotype the population. Also for this population QTL analysis was performed with mapQTL 6 using the standard interval mapping. A QTL of 27.0 cM with a LOD score of 11 was identified around 50 Mbp on chromosome 8. The QTL covers the area between 44.786.623 and 62.697.887 bp. This QTL explained 12 percent of the phenotypes.

### Conclusion Genetics No Bleed.

From the two studied mapping populations it can be concluded that the No bleed trait is polygenic. At least a recessive locus on chromosome 5 and a recessive locus on chromosome 8 are involved.

### Markers Linked to the No Bleed Trait

TABLE 2
SNPs linked to the No bleed trait in population 1.

| | | Position | Allele linked | Alternative |
|---|---|---|---|---|
| SNP | Chromosome | Chromosome* (bp) | to Nobl | allele |
| 1 | C: 5 | 189,933,999 | C | G |
| 2 | C: 5 | 191,926,614 | G | A |
| 3 | C: 5 | 192,475,871 | G | A |
| 4 | C: 5 | 192,931,982 | A | C |
| 5 | C: 5 | 193,448,175 | A | G |
| 6 | C: 5 | 194,161,015 | G | A |
| 7 | C: 5 | 195,214,599 | A | C |
| 8 | C: 5 | 195,656,147 | A | G |
| 9 | C: 5 | 196,383,956 | A | G |
| 10 | C: 5 | 200,530,017 | A | G |

*The reference genome is: V8 (Reyes-Chin-Wo et al., 2017; https://lgr.genomecenter.ucdavis.edu/)

TABLE 3
SNPs linked to the No bleed trait in population 2.

| | | Position | Allele linked | Alternative |
|---|---|---|---|---|
| SNP | Chromosome | Chromosome* (bp) | to Nobl | allele |
| 11 | C: 8 | 44,786,623 | C | T |
| 12 | C: 8 | 48,407,086 | G | A |
| 13 | C: 8 | 49,291,632 | G | T |
| 14 | C: 8 | 49,309,847 | A | G |
| 15 | C: 8 | 49,648,286 | A | G |
| 16 | C: 8 | 49,984,477 | C | T |
| 17 | C: 8 | 53,583,703 | A | G |
| 18 | C: 8 | 60,283,621 | T | C |
| 19 | C: 8 | 61,286,974 | C | T |
| 20 | C: 8 | 62,296,168 | T | C |
| 21 | C: 8 | 62,697,887 | T | C |

*The reference genome is: V8 (Reyes-Chin-Wo et al., 2017; https://lgr.genomecenter.ucdavis.edu/)

TABLE 4
Sequences used to develop KASP assays targeting the SNPs provided in table 2 and 3.
The odd SED ID Nos correspond to SNP markers associated with the presence
of the No bleed trait, in accordance with tables 2 and 3.

| | | | Position | |
|---|---|---|---|---|
| SEQ | | Chromo- | chromosome* | |
| ID No | SNP | some | (bp) | Sequence-SNP nucleotide is in brackets |
| 1 | 1 | C: 5 | 189,933,999 | GACAGCCTTTGTGACAACCCSYTTTTCTTGGTGAAAACAAACCCTTTTTT[C] |
| | | | | GTGAAAGACAATTTGTTGTGTTTTTTACTTTATGTTCTTTATTTRCTTAG |
| 2 | | | | GACAGCCTTTGTGACAACCCSYTTTTCTTGGTGAAAACAAACCCTTTTTT[G] |
| | | | | GTGAAAGACAATTTGTTGTGTTTTTTACTTTATGTTCTTTATTTRCTTAG |
| 3 | 2 | C: 5 | 191,926,614 | AMTGAGCTTTCTAACTAATAGCATAATCGTTGAAAGTAAACACATACCYT[G] |
| | | | | TAATAAATCTTCATTTATCCAAATCCTTAGCATGATCTKAATTAACATAT |
| 4 | | | | AMTGAGCTTTCTAACTAATAGCATAATCGTTGAAAGTAAACACATACCYT[A] |
| | | | | TAATAAATCTTCATTTATCCAAATCCTTAGCATGATCTKAATTAACATAT |
| 5 | 3 | C: 5 | 192,475,871 | ATATTTTCCCGAGAACTAAATTTAGACCATTAGATCAAAAAAAATGGAGG[G] |
| | | | | CAGTGATTTTTTATGGCAGTTTTATAAATAACTAGAAAAAAGAGATGGGG |
| 6 | | | | ATATTTTCCCGAGAACTAAATTTAGACCATTAGATCAAAAAAAATGGAGG[A] |
| | | | | CAGTGATTTTTTATGGCAGTTTTATAAATAACTAGAAAAAAGAGATGGGG |
| 7 | 4 | C: 5 | 192,931,982 | GGACTCCTGCTCAAGCATCACTCGCATGGGTGGCGGAACAACGGTGGTAT[A] |
| | | | | GGTATGGCTCGAATTCTTGTGGTGGAGGAAAGGAATGCGGTCACTATACA |
| 8 | | | | GGACTCCTGCTCAAGCATCACTCGCATGGGTGGCGGAACAACGGTGGTAT[C] |
| | | | | GGTATGGCTCGAATTCTTGTGGTGGAGGAAAGGAATGCGGTCACTATACA |
| 9 | 5 | C: 5 | 193,448,175 | CACCCCAAAAAMTCCAAATAATCAYCCTTCAAATCACAAAAGAAACTTTC[A] |
| | | | | TACCAATCATTCTTTGAATTTCAWTCTAAACCRATTATTAACCCTTCGAA |
| 10 | | | | CACCCCAAAAAMTCCAAATAATCAYCCTTCAAATCACAAAAGAAACTTTC[G] |
| | | | | TACCAATCATTCTTTGAATTTCAWTCTAAACCRATTATTAACCCTTCGAA |
| 11 | 6 | C: 5 | 194,161,015 | CGCAAACCRCACCGTAAAGTGCAGGATATCAAAAACATGAACCGCGAACC[G] |
| | | | | YACCAAGAATGTTYTAAACCGCATTACACGGTGCAGGCGGTTTATGTKGT |
| 12 | | | | CGCAAACCRCACCGTAAAGTGCAGGATATCAAAAACATGAACCGCGAACC[A] |
| | | | | YACCAAGAATGTTYTAAACCGCATTACACGGTGCAGGCGGTTTATGTKGT |
| 13 | 7 | C: 5 | 195,214,599 | GTAGCTTGATTCATGTGATGAGATCTTATCTATACCCTATACAGGTTGTT[A] |
| | | | | TGAATGACAATTGACTTTTATCACATCCTTATGAAAGTYCCATAGTATGT |
| 14 | | | | GTAGCTTGATTCATGTGATGAGATCTTATCTATACCCTATACAGGTTGTT[C] |
| | | | | TGAATGACAATTGACTTTTATCACATCCTTATGAAAGTYCCATAGTATGT |
| 15 | 8 | C: 5 | 195,656,147 | ATCTTGGTAGAATTCACAAGATTCATCATGACATCAACTTGTACCCTAAG[A] |
| | | | | GAAAGTATATAGTCTAATGCTTCTTTAAAGATGGTGAAATCATCCATGGC |
| 16 | | | | ATCTTGGTAGAATTCACAAGATTCATCATGACATCAACTTGTACCCTAAG[G] |
| | | | | GAAAGTATATAGTCTAATGCTTCTTTAAAGATGGTGAAATCATCCATGGC |
| 17 | 9 | C: 5 | 196,383,956 | ACATGTTCAAAATTCGGTAYGGAATTAACACGATTTAACATTGTTGTCGT[A] |
| | | | | TTTTTCGTATTTGTCGTGTTATGTATGATTARGTATTAATTAATTAAACT |
| 18 | | | | ACATGTTCAAAATTCGGTAYGGAATTAACACGATTTAACATTGTTGTCGT[G] |
| | | | | TTTTTCGTATTTGTCGTGTTATGTATGATTARGTATTAATTAATTAAACT |
| 19 | 10 | C: 5 | 200,530,017 | GGTGTAATAATTCTTTTTCACATGTAGTTTCATCTTCTTGTGGCACTTTG[A] |
| | | | | GTGATATTTTTKCTATTTGGGATCTAGATCGTATTTCTCATAAAAGGATG |
| 20 | | | | GGTGTAATAATTCTTTTTCACATGTAGTTTCATCTTCTTGTGGCACTTTG[G] |
| | | | | GTGATATTTTTKCTATTTGGGATCTAGATCGTATTTCTCATAAAAGGATG |
| 21 | 11 | C: 8 | 44,786,623 | CAAGAATAACAAGTRATCTCTAACATGGTGGAACTAATCTTCCTGGTGGC[C] |
| | | | | TGTTCTCGAAGAATCTTAGCCCTCTCTAACTCAACCTGAGCTTGTTGCCT |
| 22 | | | | CAAGAATAACAAGTRATCTCTAACATGGTGGAACTAATCTTCCTGGTGGC[T] |
| | | | | TGTTCTCGAAGAATCTTAGCCCTCTCTAACTCAACCTGAGCTTGTTGCCT |
| 23 | 12 | C: 8 | 48,407,086 | CTCATATATGYGGTCATTGTTGAGTAAGGATTGGTTAGAATTGTAGATTA[G] |
| | | | | TTGTCTTYTCTTTTCTCTTTGGTGTCTTCSATCATGATTTCTTTTGCTCY |
| 24 | | | | CTCATATATGYGGTCATTGTTGAGTAAGGATTGGTTAGAATTGTAGATTA[A] |
| | | | | TTGTCTTYTCTTTTCTCTTTGGTGTCTTCSATCATGATTTCTTTTGCTCY |
| 25 | 13 | C: 8 | 49,291,632 | TTGCCGGTTCGCATCCAAGAGGTGATGCCCAACRAGCAACTTCATCYGAA[G] |
| | | | | TTGCTCCCACTCCATAGACTCTAGATTTATAAAAAWTGACTTTTAGACCC |
| 26 | | | | TTGCCGGTTCGCATCCAAGAGGTGATGCCCAACRAGCAACTTCATCYGAA[T] |
| | | | | TTGCTCCCACTCCATAGACTCTAGATTTATAAAAAWTGACTTTTAGACCC |
| 27 | 14 | C: 8 | 49,309,847 | TATATTCATGATTAGYCGAYGRYTGKTTTGGGAAACCAGGTTRGAGGACY[A] |
| | | | | TCAGTAGGACTCGAGTGAGTCAAGATGYWGATTGAGTGAATGTCCRRCAT |
| 28 | | | | TATATTCATGATTAGYCGAYGRYTGKTTTGGGAAACCAGGTTRGAGGACY[G] |
| | | | | TCAGTAGGACTCGAGTGAGTCAAGATGYWGATTGAGTGAATGTCCRRCAT |
| 29 | 15 | C: 8 | 49,648,286 | GTTYCCTCACTRGGWTTAGTRGGTYTAAGGCACTAATGTYGGACCTTATT[A] |
| | | | | GTTATGATAGGATGTTAGGAGTTTGCATGACATTTGCATGTGTTATGTGT |
| 30 | | | | GTTYCCTCACTRGGWTTAGTRGGTYTAAGGCACTAATGTYGGACCTTATT[G] |
| | | | | GTTATGATAGGATGTTAGGAGTTTGCATGACATTTGCATGTGTTATGTGT |
| 31 | 16 | C: 8 | 49,984,477 | AGTGAGCCAATATCATGCACAAAAGCATGGAAACAAAACATATAYGTACA[C] |
| | | | | TATGTCAAGTCATGTAATCAGATCAATCCATACARCATACATCGATAACT |
| 32 | | | | AGTGAGCCAATATCATGCACAAAAGCATGGAAACAAAACATATAYGTACA[T] |
| | | | | TATGTCAAGTCATGTAATCAGATCAATCCATACARCATACATCGATAACT |
| 33 | 17 | C: 8 | 53,583,703 | CATCGTGTCTTTCAATAACTTCCACCGACTTAAAAGTTTGGTCTTTAGTT[A] |
| | | | | TCTGACTGCAAATACTGTCTTTTGCTTATCGCTAYAGTTGCAGCTTTCAA |
| 34 | | | | CATCGTGTCTTTCAATAACTTCCACCGACTTAAAAGTTTGGTCTTTAGTT[G] |
| | | | | TCTGACTGCAAATACTGTCTTTTGCTTATCGCTAYAGTTGCAGCTTTCAA |
| 35 | 18 | C: 8 | 60,283,621 | GTCATTTCATCATCAATTGCTTCTTTCCAAAATATTACATCTTGAGACCG[T] |
| | | | | RTGGCCTCTTGAAATGTCTTTGGATCATCCCCAATACTRKAAMAATWAGA |
| 36 | | | | GTCATTTCATCATCAATTGCTTCTTTCCAAAATATTACATCTTGAGACCG[C] |
| | | | | RTGGCCTCTTGAAATGTCTTTGGATCATCCCCAATACTRKAAMAATWAGA |
| 37 | 19 | C: 8 | 61,286,974 | GGTCAGATCTGATCGGRCTAAGATCTTAAGATCCTACTTGGGTCCTAAGA[C] |
| | | | | TCTAGTATAGAACCTAGTAAGATTGATATCGTTTTTYACTTCTAAAATTT |
| 38 | | | | GGTCAGATCTGATCGGRCTAAGATCTTAAGATCCTACTTGGGTCCTAAGA[T] |
| | | | | TCTAGTATAGAACCTAGTAAGATTGATATCGTTTTTYACTTCTAAAATTT |
| 39 | 20 | C: 8 | 62,296,168 | ATTTCATACCTTTTCCATGGGCTACCCCATGGTCTTCCCTTGTAGTGCCT[T] |
| | | | | GGCTACCCTGARGTCATATACACTAGTTCCATGGGATACCCCCATGGTCA |
| 40 | | | | ATTTCATACCTTTTCCATGGGCTACCCCATGGTCTTCCCTTGTAGTGCCT[C] |
| | | | | GGCTACCCTGARGTCATATACACTAGTTCCATGGGATACCCCCATGGTCA |
| 41 | 21 | C: 8 | 62,697,887 | TGATGGTTCYTCTTCAAGTGGAACCAAAGGTGTTCTTCCTACTCCCTCTA[T] |
| | | | | CAAACCTAAAGAAGCTRAATGCTTCTATTGTCATGACAAACTATATTGTC |
| 42 | | | | TGATGGTTCYTCTTCAAGTGGAACCAAAGGTGTTCTTCCTACTCCCTCTA[C] |
| | | | | CAAACCTAAAGAAGCTRAATGCTTCTATTGTCATGACAAACTATATTGTC |

*The reference genome is: V8 (Reyes-Chin-Wo et al., 2017; https://grr.ocdayis.edu/)
The abbreviations are according to IUPAC nucleotide code:
Symbol Nucleotide Base
A Adenine
C Cytosine
G Guanine
T Thymine
N A or C or G or T
M A or C
R A or G
W A o rT
S C or G
Y C or T
K G or T
V Not T
H Not G
D Not C
B Not A

### Example 4. Introduction of the Genetic Fragments Providing the No Bleed Trait from the Deposit Number NCIMB 43972

Transformation of plants using the Agrobacterium tumefaciens system is a useful way of introducing new traits into plants not having these traits. To this end molecular biology constructs harboring the genetically encoded trait according to this invention are designed and synthesized or otherwise obtained with molecular biology techniques.

These constructs harbor:
(1) The first gene encoding the No bleed trait whereby the gene is obtainable from a first genomic fragment located on chromosome 5, between positions 189.391.684 and 201.101.671 bp, and/or
(2) The second gene encoding the No bleed trait whereby the gene is obtainable from a second genomic fragment located on chromosome 8, between 44.786.623 and 62.697.887 bp.

The positions are defined relative to the V8 reference genome: V8 (Reyes-Chin-Wo et al., 2017; http://lgr.genomecenter.ucdavis.edu/).

Lettuce plants not comprising the No bleed trait are (co-)transformed with construct (1) and/or (2) by employing co-cultivation with Agrobacterium. Upon completed transformation, stable transformants are selected. These selected stable transformants are selfed sequenced and among them plants where the introduced genes are present homozygously are subsequently selected and subjected to the test as defined in Example 1, assessing the amount of juices exerted by the plant after a cut is placed to separate the lettuce head from the root system.

It is expected that the stable, homozygous transformants will:
show substantially no bleeding, where the plant is a double transformant harboring both construct (1) and construct (2);
show significantly less bleeding as compared to conventional plants, when the plant is transformed with construct (1) only;
show decreased bleeding as compared to conventional plants not when plant, when the plant is transformed with construct (2) only;
show normal bleeding characteristics (abundant bleeding) when the plants are not transformed

1. A lettuce plant having a phenotype that the lettuce head thereof does not substantially secrete exudate from the cut surface after cutting the lettuce head from the root system, the phenotype is obtainable, obtained, or is from a lettuce plant deposited as NCIMB 43972.

2. The lettuce plant according to claim 1, wherein the plant substantially comprises that traces of exudate can be observed at the cut surface.

3. The lettuce plant according to claim 1, wherein the phenotype is encoded by two or more genes.

4. The lettuce plant according to claim 1, wherein a first gene encoding the phenotype is located in a first genomic region on chromosome 5 at a position corresponding with position 189,391,684 bp to 201,101,671 bp of the lettuce reference genome V8.

5. The lettuce plant according to claim 4, wherein the first genomic region comprises one or more sequences selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17 and SEQ ID No. 19.

6. The lettuce plant according to claim 1, wherein the phenotype is genetically linked to one or more sequences selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17 and SEQ ID No. 19.

7. The lettuce plant according to claim 1, wherein a second gene encoding the phenotype is located in a second genomic region on chromosome 8 at a position corresponding with position 44,786,623 bp to 62,697,887 bp of the lettuce reference genome V8.

8. The lettuce plant according to claim 7, wherein the second genomic region comprises one or more sequences selected from the group consisting of SEQ ID No. 21, SEQ ID No. 23, SEQ ID No. 25, SEQ ID No. 27, SEQ ID No. 29, SEQ ID No. 31, SEQ ID No. 33, SEQ ID No. 35, SEQ ID No. 37, SEQ ID No. 39 and SEQ ID No. 41.

9. The lettuce plant according to claim 1, wherein the phenotype is genetically linked to one or more sequences selected from the group consisting of SEQ ID No. 21, SEQ ID No. 23, SEQ ID No. 25, SEQ ID No. 27, SEQ ID No. 29, SEQ ID No. 31, SEQ ID No. 33, SEQ ID No. 35, SEQ ID No. 37, SEQ ID No. 39 and SEQ ID No. 41.

10. The lettuce plant according to claim 1, wherein the first gene is homozygously present in the genome of the lettuce plant.

11. The lettuce plant according to claim 1, wherein the second gene is homologously present in the genome of the lettuce plant.

12. The lettuce plant according to claim 1, wherein the plant is further resistant to Xanthomonas campestris, Erwinia carotovora, Pseudomonas cichorii, Pseudomonas viridiflava, Alternaria sonchi, Bremia lactucae, Sclerotinia, Fusarium oxysporum, Pythium, Rhizorhapis suberifaciens, Lettuce Mosaic Virus (LMV), Tomato Spotted Wilt Virus (TSWV), Lettuce Big Vein Virus (LBVV), Lettuce dieback associated viruses and/or Nasonovia ribisnigri.

13. The seeds, progeny, edible parts, egg cells, callus, suspension culture, somatic embryos, clones, embryos, or plant parts of a lettuce plant according to claim 1.

14. A method for identifying a lettuce plant comprising a phenotype that lettuce head thereof does not substantially secrete exudate from the cut surface after cutting the lettuce head from the root system, the method comprising:
identifying the presence of a first gene by establishing the presence in the genome of the lettuce plant of one or more markers selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17 and SEQ ID No. 19; and/or
identifying the presence of a second gene by establishing the presence in the genome of the lettuce plant of one or more markers selected from the group consisting of SEQ ID No. 21, SEQ ID No. 23, SEQ ID No. 25, SEQ ID No. 27, SEQ ID No. 29, SEQ ID No. 31, SEQ ID No. 33, SEQ ID No. 35, SEQ ID No. 37, SEQ ID No. 39 and SEQ ID No. 41.

15. The method according to claim 14, further comprising the step of establishing the homozygous presence of the first gene and/or the second gene.

16. (canceled)

17. A nucleic acid sequence selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17 and SEQ ID No. 19, SEQ ID No. 21, SEQ ID No. 23, SEQ ID No. 25, SEQ ID No. 27, SEQ ID No. 29, SEQ ID No. 31, SEQ ID No. 33, SEQ ID No. 35, SEQ ID No. 37, SEQ ID No. 39 and SEQ ID No. 41.
